(19) United States
(12) Patent Application Publication
(10) Pub. No.: US 2021/0345887 A1
(43) Pub. Date: Nov. 11, 2021

(54) WEARABLE THERMOMETER DEVICE

(71) Applicant: Dermal Photonics Corporation, Middleton, MA (US)

(72) Inventors: David Bean, Middleton, MA (US); Zilong Huang, Malden, MA (US); Jon Moser, Ellington, CT (US)

(21) Appl. No.: 17/315,781

(22) Filed: May 10, 2021

Related U.S. Application Data
(60) Provisional application No. 63/022,975, filed on May 11, 2020.

(51) Int. Cl. A61B 5/01; G01K 13/20; G01K 1/143; G01K 1/024; A61B 5/0205; A61B 5/00

(52) U.S. Cl. CPC A61B 5/01; A61B 5/02438; G01K 1/143; G01K 1/024; A61B 5/02055; A61B 5/0008; A61B 5/0022; A61B 5/746; A61B 5/002; A61B 5/7246; A61B 5/6823; A61B 5/6831; A61B 5/6833; A61B 2562/0271; A61B 2562/08; G01K 13/20

(57) ABSTRACT

A portable, miniature, wearable health monitor device that is capable of accurate measurement of health indicators such as body temperature and heart rate which is useful in determining illness or the onset of illness in a timely way and automatically notifying the appropriate people and organizational staff to take medical action to prevent the spread of disease and protect both the individual with early intervention and also protect the organization the person is associated with through the prevention of disease proliferation within the organization.

### RELATED APPLICATIONS

This application claims the benefit of U.S. Provisional Patent Application Number 63/022,975 filed on May 11, 2020, the content of each of which is incorporated herein by reference in its entirety.

### FIELD OF THE INVENTIVE CONCEPT

Embodiments of the present inventive concepts relates generally to devices, systems, and methods for health monitoring, and more specifically, to wearable thermometer devices, systems, and methods.

### BACKGROUND OF THE INVENTION

The COVID-19 pandemic changed the way people live, work and interact with each other. Social distancing is needed and often mandated by governments to slow the spread of this virus. Other measures such as protective equipment including hand washing, face masks and gloves are regularly used even for the general public. In many areas face masks are mandated. This is all driven by the problem of inadequate testing which leads to an inadequate understanding of who has the virus and is sick as well as who is contagious with the disease and during which timeframe. This situation is not limited to COVID-19. In the 1918 influenza outbreak, the United States and other regions of the world experienced a similar pandemic that killed 50 million people. In the case of COVID-19, the death count is over 100,000 people with only a small fraction of the world population infected as of this writing.

Given the limited testing and lack of a vaccine during such events, societies are left to quarantine themselves to slow the spread and reduce the spike of hospitalizations that may overwhelm medical capacity to care for the ill. In Q1 of 2020, full societal quarantines were implemented by most countries which had a massive impact on economies around the world with comparable economic effects to the great depression. After several months of quarantines, many governments reopened their economy and commerce even before enough testing was in place and before the pandemic is considered under control. This risky reopening of the economy puts individuals at risk, but is seen a necessary to addresses the equally important problems of unemployment and other social problems and illnesses caused by prolonged quarantines of society. There is no good solution or plan as of May 2020 to allow people return to work and school while having sufficient testing and social tracking to keep the COVID-19 virus from spreading rapidly with possible spikes of infections and hospitalizations.

When people test positive, they are typically strictly quarantined and prevented from human contact. This approach is effective if infected persons and their recent circle of social contacts can be tested and quarantined as well. The quarantine time for a person typically lasts until the illness has past and the infected person tests negative and is clear of the disease. If a person gets sufficiently ill, they will be admitted to the hospital, monitored closely, and treated for complications. The monitoring of patients often consists of monitoring temperature, heart rate, respiratory rate, and various blood levels for oxygen, glucose, and more.

Absent a vaccine or immunotherapy that eradicates with the disease or the health impact of the disease, monitoring, testing and quarantining are the only viable measures to remedy any infectious health pandemic. It is critically important for any society to get back to work and school activities as well as social activates after long periods of quarantine. Unfortunately, these activities, by their very nature require close social contact giving rise to the spread of any contagious disease, especially airborne and surface-borne diseases like COVID-19 and Influenza. Ideally, before on-campus employment and on-campus school activities and the like resume, a safe and effective testing or monitoring system or systems is deployed broadly to the population to ensure people who become ill or contagious with any serious and infectious disease are quarantined and treated in a timely manner. For societies where normal economic activity is resumed despite lacking such protective measures and where disease continue to spread in a harmful way, these societies will be well served to implement broad-based monitoring, testing and treatment measures as quickly as possible to prevent a disastrous future outbreak.

### SUMMARY OF THE INVENTION

Embodiments of the present inventive concepts include a personal health monitor (or PHM) that has a small footprint, typically no longer than 40 mm, or no wider than 40 mm, or no thicker than 10 mm and no heavier than 20 grams allowing the health monitor system to be portable, wireless and mounted on the person's body for at least 4 hours at a time. In preferred embodiments, the system has one or more of the following attributes: it measures temperature accuracy of +/−0.2 degrees Fahrenheit (or +/−0.1 degrees Celsius), has visual display to indicate health status, has wireless connection to a mobile application and/or or database system to alert user and/or others of health status on a regular basis, takes regular measurement throughout the day, has a battery life of at least one month and preferably one year without charging, is waterproof, is shockproof, and has an accelerometer or press-button user interface.

The PHM system and software may indicate various health status indications on the device, in particular, a “healthy” status, a “health is of concern” status, and a “health is a serious concern or serious risk and needs attention” status. A typical embodiment designates the current health of a wearer of the device on the device itself, with two indications of either healthy or health is of concern. An alternative embodiment may have one indication such as a light and if no indicator is showing at all, the system is considered to be in the other state of indication. For example a green light may show a user's health status as healthy, and if no green light is shown, then the user's health is identified as a concerned status. Alternatively, no light may indicate health is good while a red light may indicate health is a concern. Alternatively, if two lights are used, one may be green indicating healthy and another red indicating health is a concern. Alternatively, three lights may show where green and red indicate as previously stated and yellow or a different color may indicate a caution where the health is neither deemed as good or unhealthy and may need further investigation. In an alternative embodiment, no health indication is shown on the device itself and instead health information is wirelessly transmitted electronically to another device or system of devices to give the health indication. In another embodiment, a graphical display like an E-ink, segment or graphic LCD or LED display indicates the health status which may show the temperature reading in digits and the health status in symbols or other visual, audio, and/or tactile format.

In one embodiment, a wireless transmission by Bluetooth™, Wi-Fi or other wireless means is sent to a mobile electronic device (or MED) such as a cell phone, tablet, beeper, etc and the device software and screen provides health indication on the screen and optionally by audible speaker on the device or phone indicates the health status. The MED that wirelessly receives the health data may relay or upload this data to a database by Wi-Fi, cellular connection or other data network to a database either within the organization or through the internet to a more central database. The central database may provide analysis of the health data and send information back to the MED. In another embodiment, the legal manager of the health monitor system (which is typically the end-user but may be the custodial parent of the end-user or the organizational manager of the end-user) may program or configure the software on the MED to send the end-user data to a central database and may additionally give permission for the data to be sent to other persons of interest such as family members and other organizations of interest such as the employer organization, school organization or medical organization associated with end-user.

In one embodiment, the PHM transmits data primarily to the end-user's personal MED which may then optionally transmit the data from the MED to the database. In a preferred embodiment, any MED from any end-user in the proximity of an end-user PHM may receive data from PHM and transmit this data to the database. In this embodiment, an end-user that does not have an MED, may still have his or her data uploaded to the database by using the MED of others; this is commonly referred to as “piggybacking” on another user's device. This is most beneficial in a school environment, for example, where young children may not have an MED at school with them and will require their PHM to piggyback on the MED of a teacher or administrator.

The personal health monitor (PHM) may be battery powered with simple coin or button cell commonly used in watches, or by rechargeable type cells or batteries. Alternatively, the form of the battery or cell can be chemically integrated into the structure of the package such as in layers of thin materials. The battery power can be from any of the common battery material types including silver oxide, alkaline, Li+, zinc-air, carbon-zinc, nickel cadmium, nickel metal hydride, and the like.

The preferred method of operation is performed by a health monitor device as described herein that is applied directly to the skin of an end-user typically at the beginning of the day and is worn throughout the time when the end-user will be interacting closely with other people such as in a school, work or social setting. Typically, the device is removed once the social interaction is completed or at the end of the day. In another embodiment, the device may be applied to the end-user and kept on continually throughout the day and optionally throughout the night and changed only when necessary for cleaning or updating the adhesive or during activities where the monitor would be compromised such as in a hot tub or sauna.

In another preferred embodiment, the device is required to be applied and used for all people within a common organization or group settings such as school or work organization. By ensuring that all members of a group use the PHM, protection of the entire group is supported through monitoring of all its members before and during their interaction with the group. In this embodiment, end-users who are ill or at-risk of being ill are quickly identified through the monitoring at the individual and/or organization level. Ideally the health risk is identified before an ill group member enters the group and action is taken to quarantine and treat the at-risk person without impacting the group. In addition, if a health risk arises while a group member is interacting as part of the group, the PHM immediately notifies the end-user and/or the organization to take action to treat the person and protect the group. There are strong benefits in this method of all or most end-users of a given group wearing the PHM device over long periods of time because the end-user can “set it and forget it”. In other words, if the user is always wearing the device during group interactions and all or the vast majority of group members are doing the same, then the end-user and all end-users of the group are free to go about their daily tasks within the group with some confidence that they are healthy and the people they are interacting with in their group are also healthy. This benefits the end-user and the group tremendously as they can interact more freely and with less real medical risk and associated emotional concern.

In another embodiment the PHM device is affixed with an adhesive sticker between the device and the skin using double-sided adhesive. In another embedment the device is affixed with a 1-sided adhesive sticker over the device with the adhesive patch extending beyond the edges of the device and onto the end-user skin to hold the device securely against the skin for extended or long periods of time. Optionally the sticker may allow light or sound indicators from device to transmit through the sticker. In another embodiment, the device is affixed to the skin by a strap around an area of the body such as the arm, ankle, chest, neck, leg or back. The PHM is preferably mounted underneath the armpit of the end-user as is recommended by the US Food and Drug Administration (FDA) guidelines for accurate temperature measurement. Alternatively, the device may be mounted on the forehead, or neck or arm or leg.

The device may have an operator interface via one or more buttons or magnetic switches or radio-frequency (RF) switches or an accelerometer where the end-user taps the device as the interface. The end-user may press a button to have device take an immediate health measurement and display the outcome or may tap the device quickly two or more times to drive the accelerometer to do the same function or move past an RF reader to do the same function or move past a magnet to do the same function. Any one of or more of these interface approaches may be used. The interface options in a different sequence or combination of sequences can drive other functions such as putting the device into pairing mode so it may be connected to a cell phone or similar device.

In a preferred embodiment, the device has no interface at all except the detection of health it is measuring such as temperature or heart rate. In this embodiment, when the device is mounted on the user, the device microprocessor will calculate or otherwise determine that it is on a human due to the health measurements in a reasonable range for human use, as opposed to measurements when it is in the box or otherwise not mounted on a person. In doing so, the microprocessor may communicate with one or more sensors and/or other mechanical, electrical, or electro-mechanical apparatuses that in turn collect data that establishes whether the device is mounted on, directly abutting, or proximal the user. When the device microprocessor calculates it is on a person for the first time, it may automatically enter setup mode without any other needed user interface action. Once this setup mode is triggered, the device sounds or lights may indicate such setup mode and the end-user may have an instruction manual to use a MED such as a cell phone to download a software application, connect wirelessly to the device and completed the setup. Once the device is setup, each subsequent time the device is placed on the end-user, the device may know operate normally as a monitor for that end-user with the setup already in place.

In another embodiment of the method of operation of the device measures the health indications and processes the data and any indications (lights, sounds, etc) very quickly, preferably in less than 20 milliseconds (ms), then microprocessor enters an ultra-low energy consumption state whereby the drain on the battery or cell is less than 10 microamps (μa). The device them waits a predetermined time and repeats the process of measurement and processing the measurement then going back to ultra-low energy consumption state. The benefit of this process is that it saves battery or cell power and allows for a small and low-cost battery/cell to be used while still maintaining a long battery life.

Other embodiments include another method of use, where an RF identification chip (RFID) on the device or on the end-user's person such as an RFID embedded into an employee ID, or RFID embedded into a student ID, or RFID embedded into plastic frequency operated button (FOB) such as is commonly referred to as a “key fob” or similar FOB card or plastic element, is read by and RF reader at the organization when end-user is close to or entering the organization. Upon reading of the RFID, a computer system may look up the end-user information in a database and this data can include health information including health information uploaded from the end-user's PHM. An organization system, for example, one or more computers storing and processing data regarding the organization, can then quickly determine the health status of the user, e.g., if the end-user is healthy or if there is a health risk (such as an illness determination from monitor date, a risk or illness from monitor data, a separate health concern in the organization system, or an indication of missing PHM where health is unknown), and if there is a health concern in the system, an alarm or notification can be triggered in one or more places including at the point of entry in the organization building or vehicle (such as a student entering a school bus), or on the phone/MED of one or more of the end-user, the organization administrators, custodial/family members of the end-user, or medical personnel. This timely notification of at-risk end-users entering or trying to enter the organization can allow the end-user and/or the organization and/or the custodial/family members and/or medical personnel to take quick action to get medical attention to end-user so they do not infect other people in the organization with illness and so that the end-user may be tested, quarantined and treated as needed, either by the organization or at the end-users own personal discretion. One end-user may have multiple PHMs due to lost or misplaced PHMs and this can be managed in database by the database simply checking that one of the devices registered to end-user is currently in use at the time of entrance.

In another embodiment, the PHM records Bluetooth™ signals from other nearby PHMs throughout the day and this information is uploaded to the MED and/or the database. In this embodiment, when it is determined that any end-user is ill, a query of the database can determine all the other end-users that the ill end-user was close to and possibly infected during the time the person was possibly contagious with the disease. This information can then be use as a means of social tracing of those other end-users who were likely exposed to the end-user while contagious. With this social tracing data, appropriate medical measures can be taken like quarantining and testing of people within the at-risk social tracing network of contagious end-user.

In another embodiment, each time the PHM takes a measurement it records the location either directly through an on-board location measurement circuit of the PHM device or preferably indirectly through connection to a nearby phone or other location-enabled MED where the MED's GPS location is used. The location information is then send to the PHM or preferably it is added to the heath data information from the PHM as the MED uploads the health data from the PHM to the database. In the same way, as outlined previously with the Bluetooth signals, this location data may be used for social tracing of at-risk end-users so appropriate medical measures can be taken. The location information maybe from GPS signal information and may be used by custodial/family members and/or organizations to monitor the location of end-users as needed and as is legally appropriate. This may be beneficial for the health and safety of the end-users especially if the end-user is a young child or handicapped and in need of such monitoring.

In another embodiment, the PHM information is output in the form of electronic data to a database whereby an organization or other party of interest may monitor the health of all of its constituents at over time and analyze longitudinal data. The organization can use the data to spot trends such as which health changes indicate illness in likely based on monitor data and comparing it to the actual timing of end-user illnesses. By observing large amounts of data and correlating such data to health information, and/or location information, and/or social tracing information, the organization can determine which indicators or combination of indicators lead to bad healthy outcomes as measured by sickness or illness transmission rates and which indications do not correlate to such outcomes. This approach could lead to non-obvious results that drive organizational policy which could dramatically reduce infection rates. For example, large amounts of data may determine that illness is not translated outside in warm weather due to air flow, temperature, UV light and other factors and that social distance when outside does not correlate to transmission and illness. Such analysis (although hypothetical, in this case) could drive an organization's policy to dramatically increase safety by having as much organizational meeting time done outside as possible and keeping windows open with ample fresh air ventilation and social distancing while inside.

In one aspect, a system for providing a health monitoring of an individual person by skin contact comprises a wearable device comprising: a material in contact with skin with thermal conductivity greater than 1 W/mK, a thermal sensor, a thermally conductive media capable of transmitting heat between thermal sensor and material in contact with skin, a microprocessor connected electrically to the thermal sensor, an adhesive sticker or elastic strap to affix device to person, an internet-connected device, a wireless connection between the wearable device and an internet-capable device, wherein: the accuracy of temperature measurement is within +/−0.2 degrees Fahrenheit or +/−0.1 degrees Celsius, health data such as temperature measurements are transmitted periodically from wearable device to internet-connected device, device battery or charge cell life is greater than one month under normal use, and health indication is conveyed or communicated for human notification on one or more of: the wearable device via lights or graphical display or audible sounds, the internet-connected device via a software application, a database system via upload of data from internet-connected device, an organization system where individual person wearing device is a member.

In some embodiments, the battery or electrical capacity of the device is greater than one year.

In some embodiments, the device is no larger than 40 mm by 40 mm by 10 mm in any dimension of size and no greater than 20 grams in weight.

In some embodiments, the device is no larger than 35 mm by 30 mm by 8 mm in any dimension of size and no greater than 10 grams in weight.

In some embodiments, multiple wearable devices connect to a single internet-connected device.

In some embodiments, the device recognizes when it is in contact with the human skin based on health measurements which are in a reasonable range for human operation as compared to when device is in box or not in contact with live human skin, wherein when the microprocessor if first placed in contact with skin, the device goes into setup mode with internet-connected device and thereafter the device works in normal measurement operation mode when in contact with human skin.

In some embodiments, the material in contact with skin has a thermal conductivity greater than 10 W/mK.

In some embodiments, the device waterproof to IP67 rating or higher.

In some embodiments, the device waterproof to IP68 rating.

In some embodiments, system further comprises a radio frequency identification chip (RFID) in the possession of the individual wearing device wherein the RFID chip is part of device, or part of person's student or employment ID card, or within the person's possession as a RFID tag or as a frequency operated button (FOB), and when person attempts to physically enter onto organization's campus or facility the RFID is automatically read using by an RFID reader and the RFID data is used to look up the person's health records including at least some data or analysis of data uploaded from one of person's devices.

In some embodiments, an organization or organization's system makes a determination if person is allowed to enter campus freely or alternatively is not allowed to enter campus freely and optionally an alarm or notification is be alerted by one or more of: the access point physical location (such as with an alarm sound and/or lights), the organization database, the organization software or web portal system to health data, the organization administrator(s) personal electronic devices, the end-user personal electronic device(s), the end-user custodial/family member(s) personal electronic device(s), email to any of these people, and text messages to any of these people.

In some embodiments, the device records Bluetooth™ signals from other nearby devices of the same type and logs this data throughout the day, and wherein this data is accessible to determine which other people this person has been in close proximity to over time.

In some embodiments, the device records the location either directly through an on-board location measurement circuit such as GPS within the device or indirectly by recording the location via the wireless connection to a nearby internet-connect device which has location measurement capability and wherein this data is accessible to determine which other people this person has been in close proximity to over time.

In some embodiments, the system further comprises vias in the control board under the thermal sensor which are filled with thermally conductive material with thermal coefficient greater than 1 W/mK.

In some embodiments, the system further comprises vias in the control board under the thermal sensor which are filled with thermally conductive material with thermal coefficient greater than 10 W/mK.

In some embodiments, the device is mounted underneath the armpit for accurate temperature measurement.

In some embodiments, the device is mounted on the arm, ankle, chest, neck, leg or back.

In some embodiments, the system further comprises a heart rate monitor or EKG measurement.

In some embodiments, device health data is uploaded to a central database and analysis may be performed to correlate actual human health measurements to which persons became ill and determine the likely limits or more precise limits of measured data that correspond to people when they are ill and/or when they may be infection with a given illness.

In some embodiments, the temperature sensor is a silicon bandgap temperature sensor.

In some embodiments, the temperature sensor is mounted on a printed circuit board and heat is conducted from skin to the device material in contact with skin to a thermal media of paste or epoxy or grease to the backside of the printed circuit board through the printed circuit board and to the thermal sensor mounted on the printed circuit board.

In some embodiments, the system further comprises vias in the control board under the thermal sensor which are filled with thermally conductive material with thermal coefficient greater than 1 W/mK.

In some embodiments, the system further comprises vias in the control board under the thermal sensor which are filled with thermally conductive material with thermal coefficient greater than 10 W/mK.

In some embodiments, the system is monitored by an organization where the end-user is a member whereby the organization can monitor and/or be altered at any moment when end-user or any end-user member within its organization becomes ill or one or more signs of health concern as determined or indicated using heath data in organization system or database wherein such information may be acted upon by the organization or person(s) within organization to take action to treat, quarantine or otherwise manage health concern.

In another aspect, a system for providing a health monitoring of an individual person by skin contact comprises: a wearable device comprising: a material in contact with skin with thermal conductivity greater than 1 W/mK; a thermal sensor mounted on a control board; a thermally conductive media capable of transmitting heat between the thermal sensor and the material in contact with the skin either directly or indirectly to where the thermal sensor is mounted on the control board; a microprocessor connected electrically to the thermal sensor; an adhesive sticker or elastic strap to affix the wearable device to a person; an internet-connected device; and a wireless connection between the wearable device and the internet-connected device, wherein: the accuracy of temperature measurement is within +/−0.2 degrees Fahrenheit or +/−0.1 degrees Celsius; health data such as temperature measurements are transmitted periodically from the wearable device to internet-connected device; device battery or charge cell life is greater than one month under normal use; and health indication is conveyed or communicated for human notification on one or more of: the wearable device via lights or graphical display or audible sounds, the internet-connected device via a software application, a database system via upload of data from internet-connected device, and an organization system where the person wearing the wearable device is a member.

Under this aspect, one or more of the following embodiments include:

The battery or electrical capacity of the device is greater than one year.

The device is no larger than 40 mm by 40 mm by 10 mm in any dimension of size and no greater than 20 grams in weight.

The device is no larger than 35 mm by 30 mm by 8 mm in any dimension of size and no greater than 10 grams in weight.

Multiple wearable devices connect to a single internet-connected device.

The device recognizes when it is in contact with the human skin based on health measurements which are in a reasonable range for human operation as compared to when device is in box or not in contact with live human skin, wherein, when the microprocessor if first placed in contact with skin, the device goes into setup mode with internet-connected device and thereafter the device works in normal measurement operation mode when in contact with human skin.

The material in contact with skin has a thermal conductivity greater than 10 W/mK.

The device waterproof to IP67 rating or higher.

The device waterproof to IP68 rating.

The system further comprising a radio frequency identification chip (RFID) in the possession of the individual wearing device wherein the RFID chip is part of device, or part of person's student or employment ID card, or within the persons possession as a frequency operated button (FOB), and when person attempts to physically enter onto organization's campus or facility the RFID is automatically read using by an RFID reader and the RFID data is used to look up the person's health records including at least some data or analysis of data uploaded from one of person's devices, and wherein organization or organization's system makes a determination if person is allowed to enter campus or alternatively is not allowed to enter campus freely.

An alarm or notification will be alerted by one or more of: the access point physical location (such as with an alarm sound and/or lights), the organization database, the organization software or web portal system to health date, the organization administrator(s) personal electronic devices, the end-user personal electronic device(s), and the end-user custodial/family member(s) personal electronic device(s), email to any of these people, text to any of these people.

The device records Bluetooth signals from other nearby devices of the same type and logs this data throughout the day, and wherein this data is accessible to determine which other people this person has been in close proximity to over time.

The device records the location either directly through an on-board location measurement circuit such as GPS within the device or indirectly by recording the location via the wireless connection to a nearby internet-connect device which has location measurement and wherein this data is accessible to determine which other people this person has been in close proximity to over time.

The system of claim 1 further comprises vias in the control board under the thermal resister which are filled with thermally conductive material with thermal coefficient greater than 1 W/mK.

The system of claim 1 further comprises vias in the control board under the thermal resister which are filled with thermally conductive material with thermal coefficient greater than 10 W/mK.

A device is mounted underneath the armpit for accurate temperature measurement.

The device is mounted on the arm, ankle, chest, neck, leg or back.

The system of claim 1 further comprises a heart rate monitor or EKG measurement.

Device health data is uploaded to a central database and analysis may be performed to correlate actual human health measurements to which persons where actually ill and determine the likely limits or more precise limits that correspond to people when they are ill and/or whe they may be infection with a given illness.

The temperature sensor is a silicon bandgap temperature sensor.

The users temperature is displayed in a graphical format over time and compares their recent temperature over the past several hours to past several days to their historical temperature as derived by time-series averaging for the same hours of the day.

The system uses only the higher temperatures for a given period of time and omits the lower temperatures for a given period of time to improve the accuracy of the overall temperature display due to the fact that the lower temperatures are likely less accurate due the nature of the temperature being taken on the skin whereby lower temperatures are more likely to be in error than higher temperatures.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a perspective view of a personal medical device and adhesive assembly (not shown) mounted together on a person, in accordance with some embodiments;

FIG. 2 is the system from FIG. 1 with the laser system unmounted from the double-sided adhesive, in accordance with some embodiments;

FIG. 3 is a front perspective view illustrating the device and double-sided adhesive patch of FIG. 2 in greater detail, in accordance with some embodiments;

FIG. 4 is a back perspective view illustrating the device and double-sided adhesive patch of FIG. 2 in greater detail, in accordance with some embodiments;

FIG. 5 is a front perspective view illustrating the device of FIG. 2 and single-sided adhesive patch detail, in accordance with some embodiments;

FIG. 6 is a back perspective view illustrating the device of FIG. 2 and single-sided adhesive patch detail, in accordance with some embodiments;

FIG. 7 is a front perspective view of the device of FIG. 2;

FIG. 8 is a front perspective view of the device with LCD display in accordance with some embodiments;

FIG. 9 is an exploded view of the parts of the device of FIG. 8;

FIG. 10 is a front exploded view of the parts of the device of FIG. 7;

FIG. 11 is a back exploded view of the parts of the device of FIG. 7; and

FIG. 12 is a flow chart of a method of operation of personal medical devices and associated network systems, in accordance with some embodiments.

### DETAILED DESCRIPTION OF EMBODIMENTS

One embodiment of the present invention includes a personal health monitor (PHM) that has a small footprint, typically no larger than 40 mm by 40 mm by 10 mm on any dimension and preferably no larger than 35 mm by 30 mm by 8 mm on any dimension, and has a weight of no more than 20 grams, preferably no more than 10 grams and allowing the health monitoring to be portable, wireless and mounted on the person's body for at least 8 hours at a time and preferably greater than 24 hours at a time. The dimensions are not limited thereto, and other dimensions may equally apply. The preferred embodiment includes one or more of the following attributes: it measures temperature accuracy of +/−0.2 degrees Fahrenheit (or +/−0.1 degrees Celsius), has LEDs or graphic display to indicate health status, has wireless connection to database system to alert user and/or others of health status on a regular basis, takes regular measurement throughout the day, has a battery life of at least one month and preferably one year without charging, is waterproof to IP67 rating and preferably IP68, is shockproof, has an accelerometer or press-button user interface or magnetic sensor.

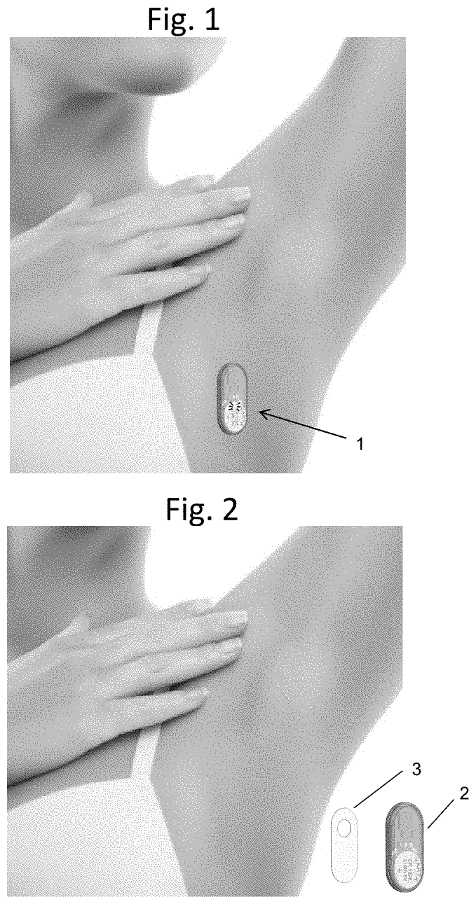

FIG. 1 illustrates the device and adhesive assembly (not shown as it is under device) mounted together as a single unitary apparatus 1 as mounted in the armpit of the end-user. FIG. 2 illustrates device (or personal medical device, PHM) 2 and double-sided adhesive part 3 just prior to mounting on end-user. The preferred method of mounting in FIG. 2 is for the end-user to stick the adhesive part 3 on the device 1 first, and then stick that assembly onto the end-user skin in the appropriate place preferably as defined by the FDA for accurate and acceptable measurements.

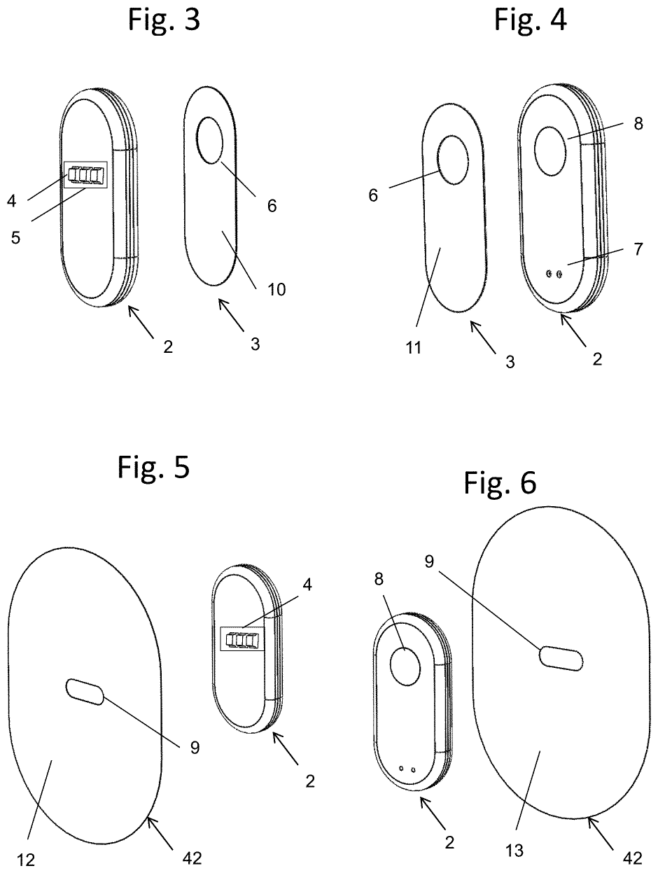

FIG. 3 illustrates the front of device 2 in more detail with window 5 for indicator lights to shine through and three indicator lights 4 in this embodiment. FIG. 3 also includes the double-sided adhesive 3 with detail showing the front of adhesive 10 which is preferably an adhesive surface that is covered by a wax-like paper which is peeled off to expose the adhesive just prior to applying to the backside of the device 2. In some embodiments, adhesive 3 has hole 6 cut into it to allow for the metal temperature probe portion of device 2 to contact skin directly. In some embodiments, the contact is indirect, e.g., via an intervening object, material, and so on.

FIG. 4 illustrates the back of device 2 in more detail with a temperature probe 8, preferably made of material with thermal conductivity greater the 1 W/mK and preferably greater than 10 W/mK such as stainless steel, and optionally ports 7 for battery recharging, or related holes or the like for exposing other components of the device 2. FIG. 4 further illustrates the back of double-sided adhesive 3 with detail showing the back of adhesive 11 which is preferably an adhesive surface that is covered by a wax-like paper which is peeled off to expose the adhesive just prior to applying the device 2 onto the end-user skin. Adhesive 3 has hole 6 cut into it to allow for the metal temperature probe 8 of device 2 to contact skin directly.

FIG. 5 illustrates the front of device 2 with single-sided adhesive 42 with detail showing the front of adhesive 12 which is preferably a non-adhesive surface that optionally may be covered by design graphics which are appealing to the end-user (such as cartoon characters for your children end-users, for example). Adhesive 42 has hole 9 cut into it to allow for the viewing of device 2 indicators 4. In an alternate embodiment, hole 9 does not exist and the indications either visibly shine through adhesive 42 or there are no indication on the device and indications occur either audibly and/or via the mobile device and/or through the database to the organization and/or apps for the custodial/family members.

FIG. 6 illustrates the back of device 2 in more detail with temperature probe 8. FIG. 6 further illustrates the back of single-sided adhesive 42 with detail showing the back of adhesive 13 which is preferably an adhesive surface that is covered by a wax-like paper which is peeled off to expose the adhesive just prior to applying onto device 2 such that hole 9 lines up with indicators 4 of FIG. 5, and then adhesive 42 in combination with device 2 is jointly applied to end-user skin with adhesive 42 edges that are wider than device 2 adhering the assembly to end-user's skin. The adhesive 42 may have other larger dimensions than device 2, such as surface area, perimeter size, and so on that permit that device 2 to be partially or completely covered or surrounded by the adhesive 42 (absent hole 9).

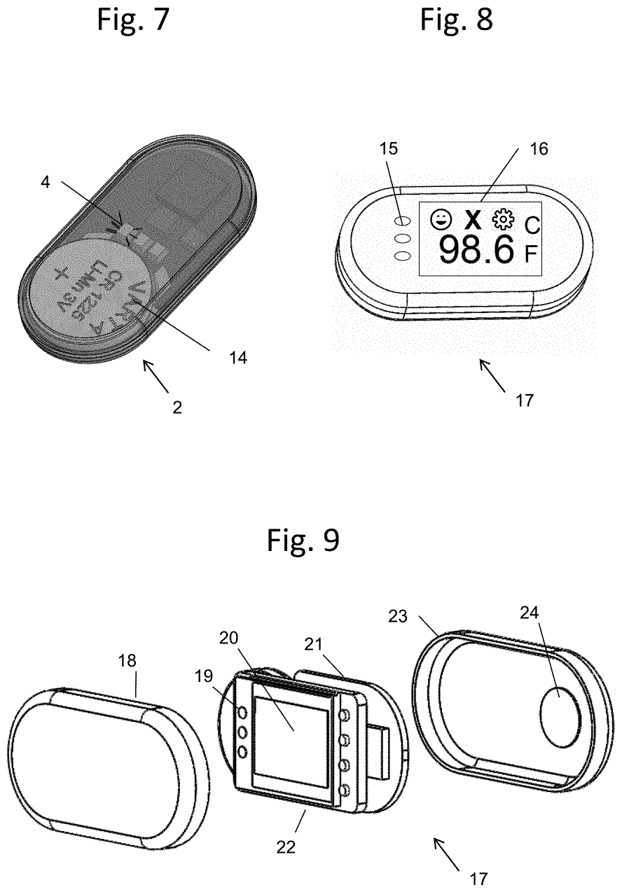

FIG. 7 illustrates the front of device 2 in more detail with clear top shell 14 acting as a window for indicator lights 4, in this embodiment. Also visible in this view top shell 14 is the button cell and electronic chips in this embodiment. In some embodiments, the entire top shell 14 is formed of a clear material such as plastic. In other embodiments, portions of the top shell 14 are clear, in particular, a region of the shell positioned over the indicator lights 4.

FIG. 8 illustrates a different embodiment where device 17 front view shows a graphical visual display indicating temperature numbers and graphical health indications 16 and optional light indicators 15 as well.

FIG. 9 illustrates an exploded view of the parts of device of FIGS. 8 with cover 18, display 20, light indicators 19, printed circuit board 21, case back 23 and skin temperature probe 24.

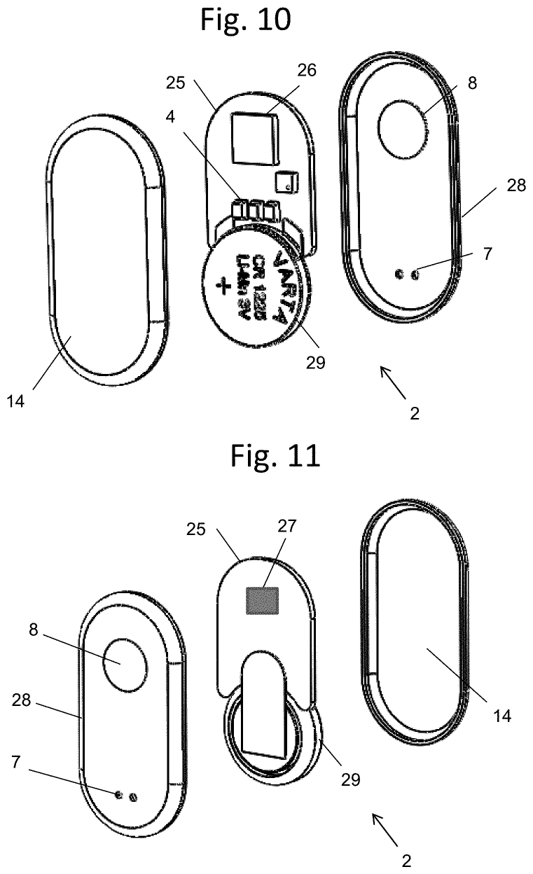

FIG. 10 illustrates the front exploded view of the parts of the device of FIG. 7 with cover 14, light indicators 4, printed circuit board 25, thermal sensor 26 (the electrical element that indicates temperature and is wired to signal the microprocessor), battery/button cell 29, case back 28, battery recharging pins 7, and skin temperature probe 8. The cover 14 and case back 28 are constructed and arranged for collectively positioned about the printed circuit board 25 and battery/button cell 29. A preferential attribute of this embodiment is to have highly thermal conducting vias (or through elements, which are typically metal such a copper; see 27 of FIG. 11) in the printed circuit board 25 under the thermal sensor 26 and thermal media (e.g., paste or epoxy or thermal grease or similar) is added onto the inside of temperature probe 8 such that when device 2 is assembled, the thermal paste physically connects the temperature probe 8 to the thermal vias (27 of FIG. 11) under thermal sensor 26 such that the heat from the end-user efficiently travels from the skin through the thermal probe 8 through the thermal paste through the vias (27 of FIG. 11) to the thermal resister 26. Alternatively, the printed circuit board 25 may be made of highly thermally conductive material and this may be used instead of the vias under the thermal sensor 26 may be a thermal resistor element or preferably an integrated thermal sensor such as a silicon bandgap temperature sensor. Thermal conductivity of material in vias is greater than 1 W/mK and preferably greater than 10 W/mK.

FIG. 11 illustrates the back exploded view of the parts of the device of FIG. 7 with cover 14, printed circuit board 25, thermal vias 27 (the hole or holes through the printed circuit board that are filled with metal or highly thermally conductive material), battery/button cell 29, case back 28, battery recharging pins 7, and skin temperature probe 8.

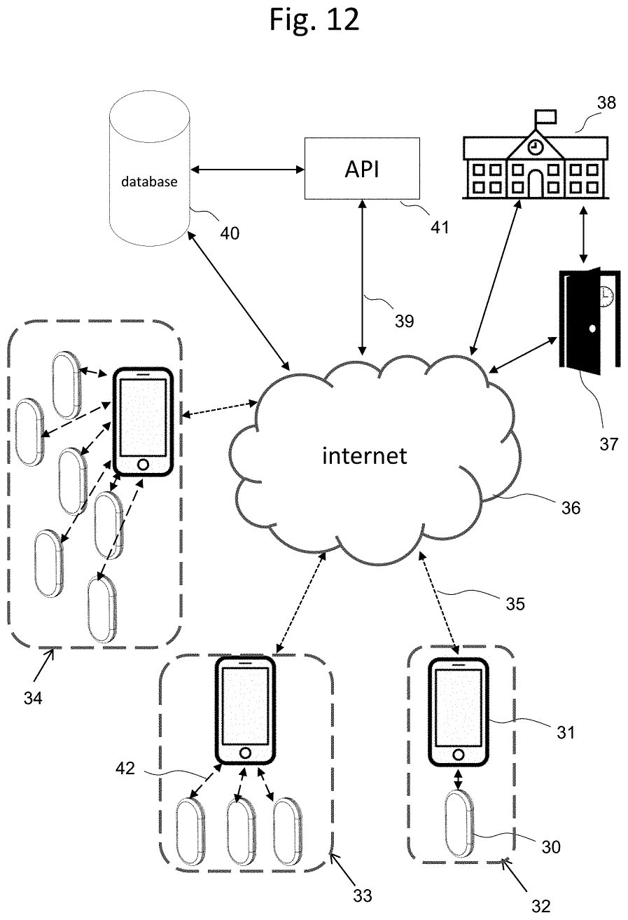

FIG. 12 is a flow chart of a method for administering PHM devices in the context of an organization that manages a group of end-users with the intent to provide some level of safety through the monitoring and management of PHMs. End-user personal heath monitors (PHMs) are shown using symbol 30, mobile electronic devices (MEDs), such as cell phones are shown with symbol 31. Communications between the PHMs 30 and MEDs 31 are shown in dashed lines 42. Communications between MEDs 31 and the internet 36 are shown in dotted lines 35. Communications between the internet 36 and the database 40 and an organization systems' application programming interface (API) 41, organization 38, and access points 37) are shown in solid lines 39. Groups of many devices 30 may connect through a single MED 31 such as in grouping 33 and grouping 34 or a single device 30 may connect through the end-user's personal MED 31 as in the one-to-one pairing in 32. In the preferred method, groupings many devices 30 may connect through one MED 31 as shown in groupings 33 and 34. It is highly preferable that all data communications are done with appropriate prior legal permission and data is encrypted and transmitted in a secure way. In the preferred method, the internet 36 is used to connect device data from MEDs 31 to the database 40. Likewise, the database may communicate back down to the device using the same pathway to send updated operating parameters or software/firmware to the devices 30. Similarly, the application or “app” on the MED 31 may be updated from the database (or app provider such as Google Play or Apple App Store). In addition, database may update parameters in the device 30 (transmitting through MED 31) and/or MED 31 such as health information, warning language, parameters to determine when warnings are triggered, device firmware, and historical data. An application program interface (API) 41 is used for the organization 38 to access data from the database to monitor constituents of the organization who have given permission for organization to access such data or for which organization has legal right to view data. API 41 may also be a website portal with security and login that provides a user interface to administrators of the organization 38 to see the status and history and data of end-users from its organization. Physical access points to the organizations such as door entries 37 may be equipped with RFID readers (or similar technology) which read end-user identification data (such as student ID number or employee ID number) as the end-user enters or approaches the organization. The access point 37 preferably relays the end-user ID information and the access location to the organization system 38 and/or database 40 where the person ID is matched up with that person's end-user PHM health status from the database (either directly or through API) and if end-user/person entering access point is deemed by database (or organization system) to be a health risk, then an alarm or notification will be alerted by one or more of: the access point physical location (such as with an alarm sound and/or lights), the organization API, the organization web portal, the organization administrator(s) MEDs, the end-user MED, and the end-user custodial/family member(s) MEDs. Email and text notification may also be send to any of the persons mentioned.

It is to be understood that the foregoing description is intended to illustrate and not to limit the scope of the invention, which is defined by the scope of the appended claims. Other embodiments are within the scope of the following claims.

What is claimed is:

1. A system for providing a health monitoring of an individual person by skin contact, the system comprising:
a wearable device comprising:
a material in contact with skin with thermal conductivity greater than 1 W/mK;
a thermal sensor mounted on a control board;
a direct physical connection or connection via thermally conductive media between the thermal sensor and the material in contact with the skin either directly or indirectly to where the thermal sensor is mounted on the control board;
a microprocessor connected electrically to the thermal sensor;
an adhesive sticker or elastic strap to affix the wearable device to a person;
an internet-connected device; and
a wireless connection between the wearable device and the internet-connected device, wherein:
the accuracy of temperature measurement is within +/−0.2 degrees Fahrenheit or +/−0.1 degrees Celsius;
health data such as temperature measurements are transmitted periodically from the wearable device to internet-connected device; and
a health indication is conveyed or communicated for human notification on one or more of: the wearable device via lights or graphical display or audible sounds, the internet-connected device via a software application, a database system via an upload of data from internet-connected device, and an organization system where the person wearing the wearable device is a member.

2. The system of claim 1, wherein the battery or electrical capacity of the device is greater than one year.

3. The system of claim 1, wherein the device is no larger than 40 mm by 40 mm by 10 mm in any dimension of size and no greater than 20 grams in weight.

4. The system of claim 1, wherein the device is no larger than 35 mm by 30 mm by 8 mm in any dimension of size and no greater than 10 grams in weight.

5. The system of claim 1, wherein multiple wearable devices connect to a single internet-connected device.

6. The system of claim 1, wherein the device recognizes when it is in contact with the human skin based on health measurements which are in a reasonable range for human operation as compared to when device is in box or not in contact with live human skin, wherein, when the microprocessor if first placed in contact with skin, the device goes into setup mode with internet-connected device and thereafter the device works in normal measurement operation mode when in contact with human skin.

7. The system of claim 1, wherein the material in contact with skin has a thermal conductivity greater than 10 W/mK.

8. The system of claim 1, wherein the device waterproof to IP67 rating or higher.

9. The system of claim 1, wherein the device waterproof to IP68 rating.

10. The system of claim 1, further comprising a radio frequency identification chip (RFID) in the possession of the individual wearing the device wherein the RFID chip is part of device, or part of a student or employment ID card of the person, or within the person's possession as a frequency operated button (FOB), and when the person attempts to physically enter onto organization's campus or facility the RFID is automatically read using by an RFID reader and the RFID data is used to look up the person's health records including at least some data or analysis of data uploaded from one of the person's devices, and wherein organization or organization's system makes a determination if the person is allowed to enter campus or alternatively is not allowed to enter campus freely.

11. The system of claim 10, wherein an alarm or notification will be alerted by one or more of: the access point physical location (such as with an alarm sound and/or lights), the organization database, the organization software or web portal system to health date, the organization administrator(s) personal electronic devices, the end-user personal electronic device(s), and the end-user custodial/family member(s) personal electronic device(s), email to any of these people, text to any of these people.

12. The system of claim 1, wherein the device records Bluetooth signals from other nearby devices of the same type and logs this data throughout the day, and wherein this data is accessible to determine which other people the person has been in close proximity to over time.

13. The system of claim 1, wherein the device records the location either directly through an on-board location measurement circuit such as GPS within the device or indirectly by recording the location via the wireless connection to a nearby internet-connect device which has location measurement and wherein this data is accessible to determine which other people this person has been in close proximity to over time.

14. The system of claim 1, further comprising:
vias in the control board under the thermal resister which are filled with thermally conductive material with thermal coefficient greater than 1 W/mK.

15. The system of claim 1, further comprising:
vias in the control board under the thermal resister which are filled with thermally conductive material with thermal coefficient greater than 10 W/mK.

16. The system of claim 1, wherein device is mounted underneath the armpit for accurate temperature measurement.

17. The system of claim 1, wherein device is mounted on the arm, ankle, chest, neck, leg or back.

18. The system of claim 1, further comprising: a heart rate monitor or EKG measurement.

19. The system of claim 1, where device health data is uploaded to a central database and analysis may be performed to correlate actual human health measurements to which persons where actually ill and determine the likely limits or more precise limits that correspond to people when they are ill and/or whe they may be infection with a given illness.

20. The system of claim 1, wherein the temperature sensor is a silicon bandgap temperature sensor.

21. The system of claim 1, wherein the users temperature is displayed in a graphical format over time and compares their recent temperature over the past several hours to past several days to their historical temperature as derived by time-series averaging for the same hours of the day.

22. The system of claim 1, wherein the system uses only the higher temperatures for a given period of time and omits the lower temperatures for a given period of time to improve the accuracy of the overall temperature display due to the fact that the lower temperatures are likely less accurate due the nature of the temperature being taken on the skin whereby lower temperatures are more likely to be in error than higher temperatures.

23. The system of claim 1, wherein a device battery or charge cell life is greater than one month under normal use;
